(19) United States
(12) Patent Application Publication
(10) Pub. No.: US 2010/0170107 A1
(43) Pub. Date: Jul. 8, 2010

(54) PLANTAR BALANCER

(76) Inventor: Tzann-Yuh TZENG, Taipei (TW)

Correspondence Address: LAW OFFICES OF KHALILIAN SIRA, LLC, 9100 PERSIMMON TREE ROAD, POTOMAC, MD, 20854, US

(21) Appl. No.: 12/575,809

(22) Filed: Oct. 8, 2009

(30) Foreign Application Priority Data
Jan. 8, 2009 (TW) 098100456

(51) Int. Cl. A43B 13/18; A43B 13/38; A61F 5/14

(52) U.S. Cl. 36/28; 36/43; 36/140

(57) ABSTRACT

The plantar balancer in accordance with the present invention has a body and a primary adjuster. The body is for padding the sole of the foot and made from a cushioning material and has a first metatarsal portion, a fourth metatarsal portion and a heel portion. The primary adjuster is positioned between the first metatarsal portion and the fourth metatarsal portion.

### BACKGROUND OF THE INVENTION

1. Field of the Invention

The present invention relates to a plantar balancer encouraging erect posture over long periods, especially to a plantar balancer implemented as an insole or a midsole of a shoe.

2. Description of the Prior Art

With reference to FIG. 16, to maintain erect posture, body weight should be distributed at a first metatarsal portion (91) around the first metatarsal bone, a fourth metatarsal portion (94) around the fourth metatarsal bone and a heel portion (96) around the heel. The three portions (91, 94, 96) constitute a three-point-supporting structure for stable support.

However, a first specific portion between the first metatarsal portion (91) and the fourth metatarsal portion (94) tends to protrude more than the metatarsal portions (91, 94). Thereby causing pressure distribution to be biased to the first metatarsal portion (91) or to the fourth metatarsal portion (94), which disrupts the aforementioned three-point-supporting structure.

Some persons unconsciously distribute their body weight to a secondary specific portion (98) between the fourth metatarsal portion (94) and the heel portion (96) over the fourth metatarsal portion (94) and the heel portion (96), which results into walking with toes turned out or walking pigeon-toed.

To overcome the shortcomings, the present invention provides a plantar balancer to mitigate or obviate the aforementioned problems.

### SUMMARY OF THE INVENTION

The main objective of the invention is to provide a plantar balancer maintaining a three-point-supporting structure to stably support human body.

The plantar balancer in accordance with the present invention has a body and a primary adjuster. The body is for padding the sole of a foot and made from a cushioning material and has a first metatarsal portion, a fourth metatarsal portion and a heel portion. The primary adjuster is positioned between the first metatarsal portion and the fourth metatarsal portion.

Other objectives, advantages and novel features of the invention will become more apparent from the following detailed description when taken in conjunction with the accompanying drawings.

### BRIEF DESCRIPTION OF THE DRAWINGS

FIG. 1 is a top view of a plantar balancer in accordance with the present invention;

FIG. 2 is a bottom view of the plantar balancer in FIG. 1;

FIG. 3 is an operational top view of the plantar balancer in FIG. 1;

FIG. 4 is an operational sectional side view of the plantar balancer in FIG. 1;

FIGS. 5-11 are partial side views of second to eighth embodiments of a plantar balancer in accordance with the present invention;

FIG. 12 is a top view of a ninth embodiment of a plantar balancer in accordance to the present invention;

FIG. 13 is an operational top view of the plantar balancer in FIG. 12;

FIG. 14 is a top view of a tenth embodiment of a plantar balancer in accordance to the present invention;

FIG. 15 is an operational top view of the plantar balancer in FIG. 14;

FIG. 16 is a bottom view of a sole of a foot in accordance with the prior art.

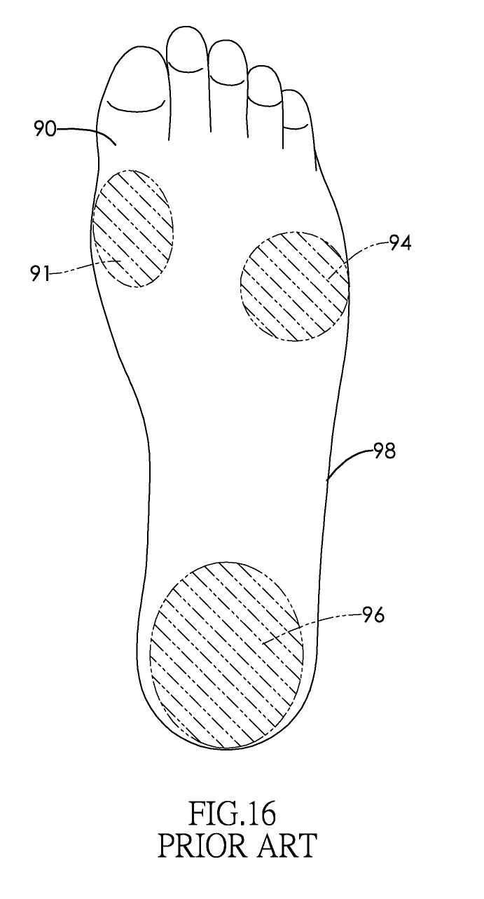

### DETAILED DESCRIPTION OF THE PREFERRED EMBODIMENTS

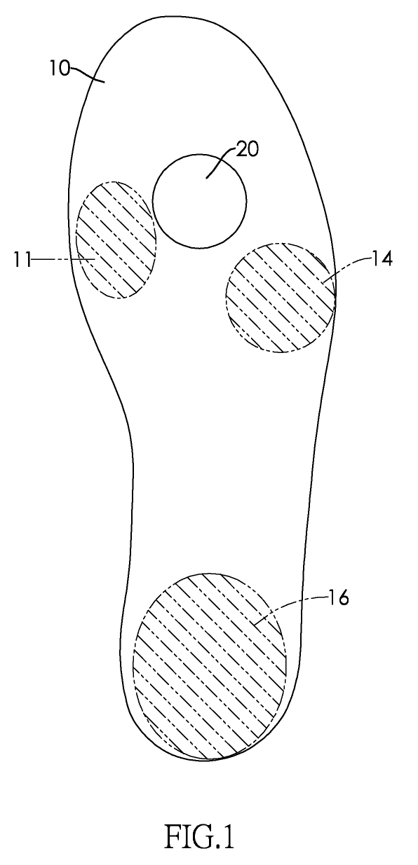

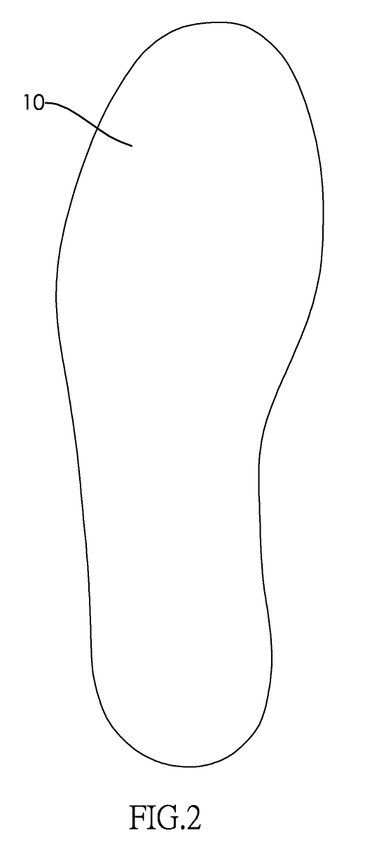

With reference to FIGS. 1 and 2, a plantar balancer in accordance with the present invention comprises a body (10) and a primary adjuster (20).

The body (10) is for padding the sole of a foot and is made from a cushioning material comprising a top, a bottom, a first metatarsal portion (11), a fourth metatarsal portion (14) and a heel portion (16). The first metatarsal portion (11), the fourth metatarsal portion (14) and the heel portion (16) are correspond respectively to a first metatarsal bone, a fourth metatarsal bone and a heel of the foot. The body (10) may be an insole for padding the sole of the foot or a midsole attached to an inner surface of an outsole of a shoe.

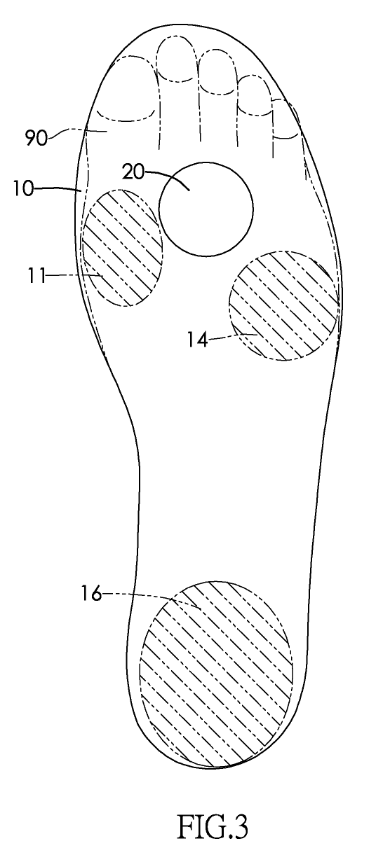

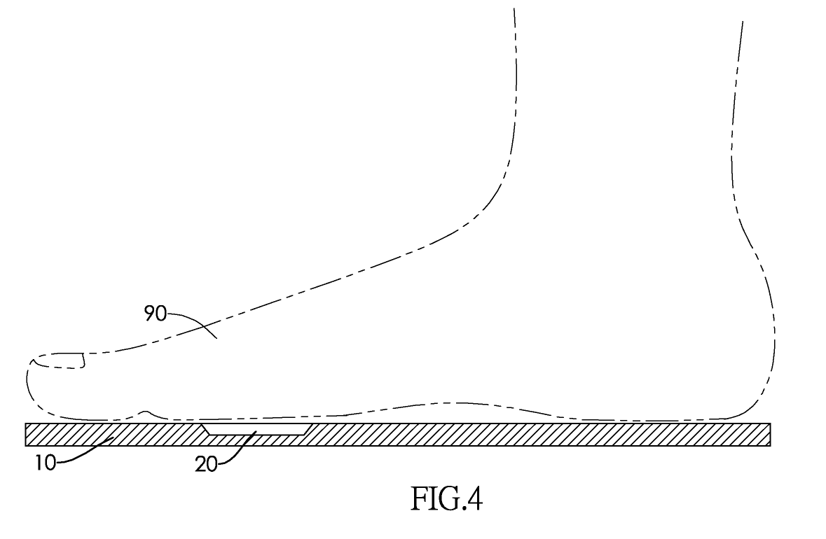

The primary adjuster (20) is positioned between the first metatarsal portion (11) and the fourth metatarsal portion (14). With reference to FIGS. 3 and 4, the primary adjuster (20) may have a structure of a concavity recessed into the top of the body (10).

When using the plantar balancer, the body (10) is placed in a shoe for padding the foot. The first metatarsal portion (11) and the fourth metatarsal portion (14) contact and cushion the portions of the foot around the first metatarsal bone and the fourth metatarsal bone. The primary adjuster is a concavity and does not solidly contact and support a first specific portion of the foot between the first metatarsal portion (11) and the fourth metatarsal portion (14). Distribution of pressure applied to the sole through the foot will be biased to the first metatarsal portion (11) and the fourth metatarsal portion (14). In other words, more pressure is distributed to the first metatarsal portion (11) and the fourth metatarsal portion (14) take more pressure than the portion between the first metatarsal portion (11) and the fourth metatarsal portion (14). Thus the first metatarsal portion (11), the fourth metatarsal portion (14) and the heel portion (16) form a three-point-supporting structure to stably support the foot. The three-point-supporting structure is formed since the primary adjuster (20) does not solidly contact the sole of the foot as well as the first metatarsal portion (11) and the fourth metatarsal portion (14).

A flipped plantar balancer of the aforementioned first embodiment resembles a plantar balancer comprising a primary adjuster (20) having a structure of a concavity recessed into the bottom of the body (10) and having a roof arcing towards the top of the body (10).

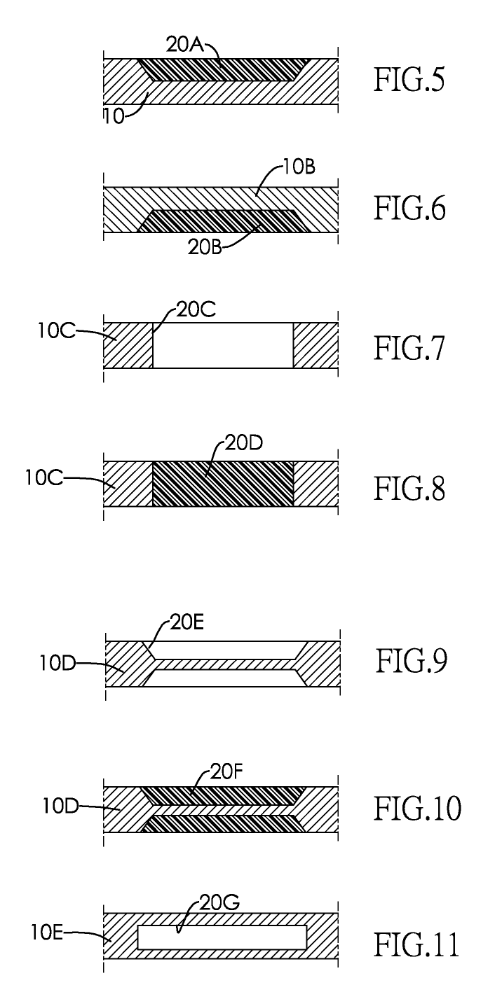

With reference to FIG. 5, a primary adjuster (20A) of a second embodiment of the plantar balancer has a structure of a set-in soft member formed into the top of the body (10), wherein the soft member is softer than the cushioning material used to make the body (10).

With reference to FIG. 6, a third embodiment of the plantar balancer has a body (10B) and a primary adjuster (20B). A concavity is recessed into the bottom of the body (10B). The primary adjuster (20B) has a structure of the aforementioned set-in soft member formed into the bottom of a body (10B). Preferably, the soft member of the primary adjuster (20B) is formed into the concavity of the body (10B).

With reference to FIG. 7, a fourth embodiment of the plantar balancer has a body (10C) and a primary adjuster (20C), wherein the primary adjuster (20C) has a structure of a hole formed in the bottom of a body (10C). With reference to FIG. 8, a primary adjuster (20D) of a fifth embodiment of the plantar balancer has a structure of the aforementioned soft member inserted in the body (10C). Preferably, the soft member of the primary adjuster (20D) is inserted in the hole of the body (10C).

With reference to FIG. 9, a sixth embodiment of the plantar balancer has a body (10D) and a primary adjuster (20E), wherein the primary adjuster (20E) has a structure of a film thinner than the body (10D). With reference to FIG. 10, a primary adjuster (20F) of a seventh embodiment of the plantar balancer has a structure of the aforementioned set-in soft members respectively formed into both the top and the bottom of the body (10D).

With reference to FIG. 11, an eighth embodiment of the plantar balancer has a body (10E) and a primary adjuster (20G), wherein the primary adjuster (20G) has a hollow structure. The aforementioned soft member may be filled in the hollow structure of the primary adjuster (20G). With the use of the aforementioned soft member, a primary adjuster (20A, 20B, 20D, 20F) provides a soft touch for the aforementioned first specific portion of the foot so that less pressure is distributed to the first specific portion than the portions around the first and fourth metatarsal bones. As a result, the pressure applied to the foot during standing, walking or running is primarily distributed at the first metatarsal portion (11), the fourth metatarsal portion (14) and the heel portion.

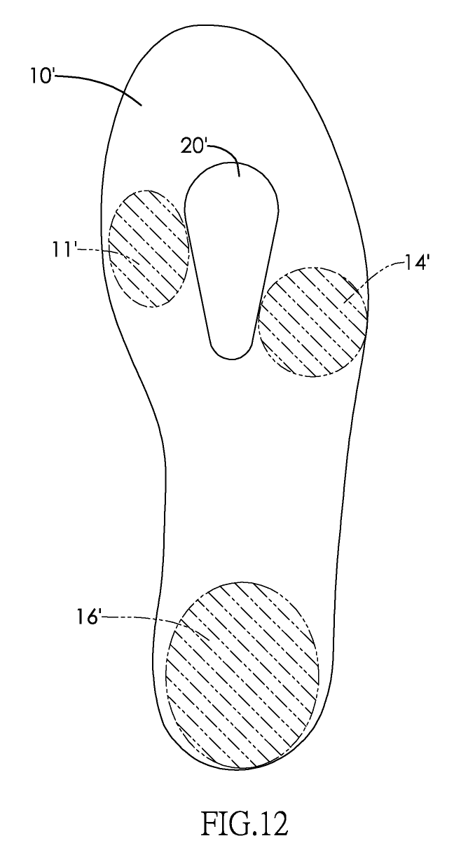

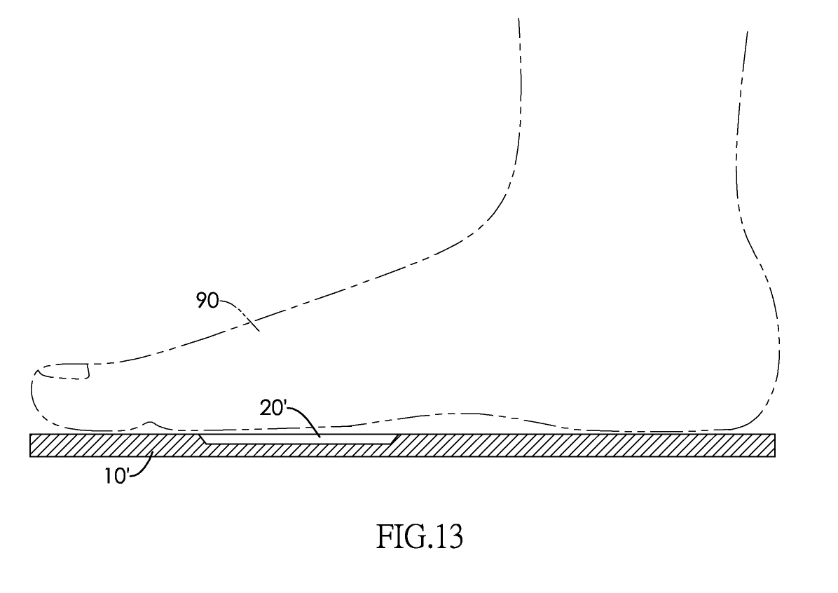

With reference to FIGS. 12 and 13, the ninth embodiment of the plantar balancer has a body (10′) and a primary adjuster (20′). The body (10′) has a first metatarsal portion (11′) and a fourth metatarsal portion (14′). The primary adjuster (20′) is elongated towards the heel portion (16′) and reaches a portion of the body (10′) corresponding to a front edge of a longitudinal arch of the foot. The primary adjuster (20′) has a structure selected from the group consisting of a structure of a hole, a structure of a soft member softer than the cushioning material inserted in the body, a structure of a concavity recessed into the top of the body, a structure of a concavity recessed into the bottom of the body, a structure of a set-in soft member softer than the cushioning material formed into the top of the body, a structure of a set-in soft member softer than the cushioning material formed into the bottom of the body, a structure of a film thinner than the body, a structure of set-in soft members softer than the cushioning material respectively formed into both the top and the bottom of the body, a hollow structure and a structure of filled-in soft members softer than the cushioning material inside the body.

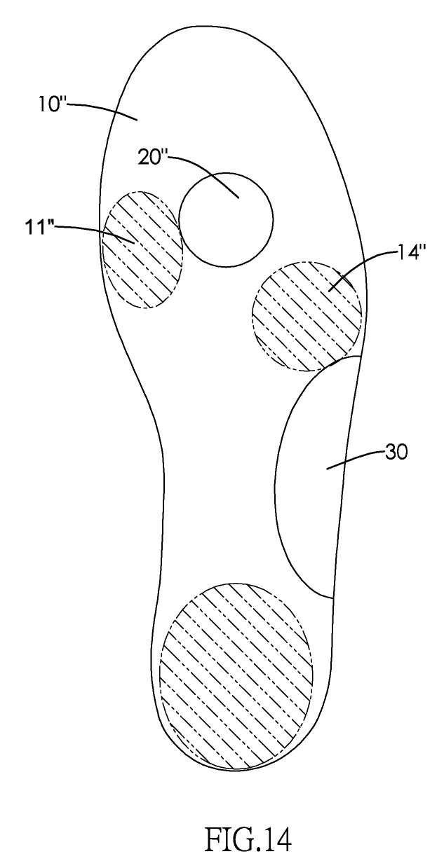

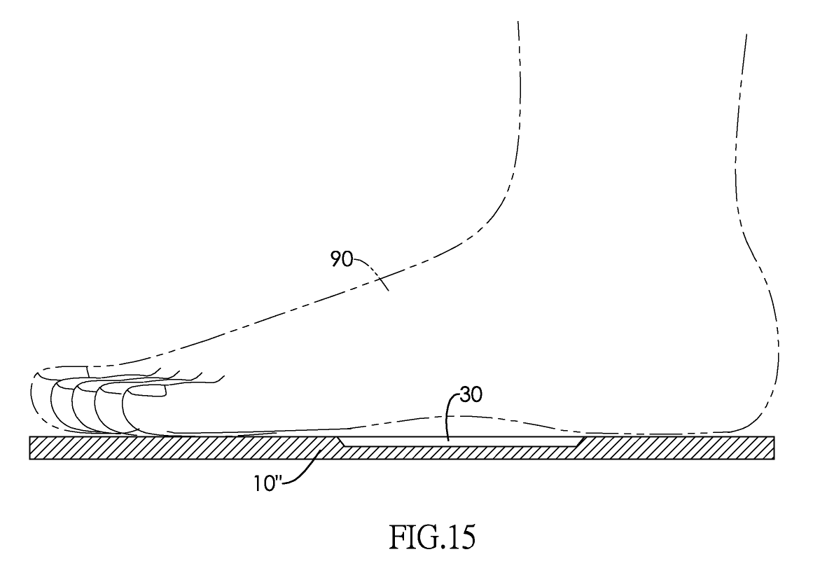

With reference to FIGS. 14 and 15, the tenth embodiment of the plantar balancer has a body (10″), a primary adjuster (20″) and a secondary adjuster (30). The body (10″) has a first metatarsal portion (11″), a fourth metatarsal portion (14″) and a heel portion (16″). The primary adjuster (20″) may have a structure as described in the aforementioned embodiments and may elongate towards the heel portion (16′) and reach a portion of the body (10′) corresponding to the front edge of the longitudinal arch.

The secondary adjuster (30) is positioned between the fourth metatarsal portion (14″) and the heel portion (16″) and has a structure selected from the group consisting of a structure of a hole, a structure of a soft member softer than the cushioning material inserted in the body, a structure of a concavity recessed into the top of the body, a structure of a concavity recessed into the bottom of the body, a structure of a set-in soft member softer than the cushioning material formed into the top of the body, a structure of a set-in soft member softer than the cushioning material formed into the bottom of the body, a structure of a film thinner than the body, a structure of set-in soft members softer than the cushioning material respectively formed into both the top and the bottom of the body, a hollow structure and a structure of filled-in soft members softer than the cushioning material inside the body.

With the aforementioned structure of the secondary adjuster (30), a second specific portion between the fourth metatarsal bone and the heel of the foot will not be supported by and not solidly contact the secondary adjuster (30). Thus in a region corresponding to the fourth metatarsal bone and the heel of the foot, pressure due to standing, walking or running is primarily distributed to the fourth metatarsal portion (14″) and the heel portion (16″). Such function of the secondary adjuster (30) helps to prevent users from standing or walking with their toes turned out.

The plantar balancer allows pressure to be distributed primarily to the first metatarsal portion, the fourth metatarsal portion and the heel portion to form a three-point-supporting structure supporting the foot stably.

What is claimed is:

1. A plantar balancer comprising
a body for padding the sole of a foot, made from a cushioning material and comprising
a top;
a bottom;
a first metatarsal portion corresponding to a first metatarsal bone of a foot;
a fourth metatarsal portion corresponding to a fourth metatarsal bone of the foot;
a heel portion for corresponding to a heel of the foot;
a primary adjuster positioned between the first metatarsal portion and the fourth metatarsal portion and having a structure selected from the group consisting of a structure of a hole, a structure of a soft member softer than the cushioning material inserted in the body, a structure of a concavity recessed into the top of the body, a structure of a concavity recessed into the bottom of the body, a structure of a set-in soft member softer than the cushioning material formed into the top of the body, a structure of a set-in soft member softer than the cushioning material formed into the bottom of the body, a structure of a film thinner than the body, a structure of set-in soft members softer than the cushioning material respectively formed into both the top and the bottom of the body, a hollow structure and a structure of filled-in soft members softer than the cushioning material inside the body.

2. The plantar balancer as claimed in claim 1, wherein the primary adjuster elongates towards the heel portion and reaches a portion of the body corresponding to a front edge of a longitudinal arch of the foot.

3. The plantar balancer as claimed in claim 1 further comprising a secondary adjuster positioned between the fourth metatarsal portion and the heel portion and having a structure selected from the group consisting of a structure of a hole, a structure of a soft member softer than the cushioning material inserted in the body, a structure of a concavity recessed into the top of the body, a structure of a concavity recessed into the bottom of the body, a structure of a set-in soft member softer than the cushioning material formed into the top of the body, a structure of a set-in soft member softer than the cushioning material formed into the bottom of the body, a structure of a film thinner than the body, a structure of set-in soft members softer than the cushioning material respectively formed into both the top and the bottom of the body, a hollow structure and a structure of filled-in soft members softer than the cushioning material inside the body.

4. The plantar balancer as claimed in claim 2 further comprising a secondary adjuster positioned between the fourth metatarsal portion and the heel portion and having a structure selected from the group consisting of a structure of a hole, a structure of a soft member softer than the cushioning material inserted in the body, a structure of a concavity recessed into the top of the body, a structure of a concavity recessed into the bottom of the body, a structure of a set-in soft member softer than the cushioning material formed into the top of the body, a structure of a set-in soft member softer than the cushioning material formed into the bottom of the body, a structure of a film thinner than the body, a structure of set-in soft members softer than the cushioning material respectively formed into both the top and the bottom of the body, a hollow structure and a structure of filled-in soft members softer than the cushioning material inside the body.

5. The plantar balancer as claimed in claim 1, wherein the primary adjuster has a structure of a concavity recessed into the bottom of the body and having a roof arcing towards the top of the body.

6. The plantar balancer as claimed in claim 1, wherein the body is a midsole.

7. The plantar balancer as claimed in claim 1, wherein the body is an insole.

8. The plantar balancer as claimed in claim 5, wherein the body is a midsole.

9. The plantar balancer as claimed in claim 5, wherein the body is an insole.
